(19) United States
(12) Patent Application Publication
(10) Pub. No.: US 2024/0188867 A1
(43) Pub. Date: Jun. 13, 2024

(54) Intelligent Attention Rehabilitation System

(71) Applicant: Fujian University of Traditional Chinese Medicine, Fuzhou City (CN)

(72) Inventors: Jingsong WU, Fuzhou City (CN); Lidian CHEN, Fuzhou City (CN); Jing TAO, Fuzhou City (CN)

(21) Appl. No.: 18/585,742

(22) Filed: Feb. 23, 2024

Related U.S. Application Data
(63) Continuation of application No. 17/155,002, filed on Jan. 21, 2021.

(51) Int. Cl. A61B 5/16; A61B 5/00; A61B 5/1171; G16H 10/20; G16H 50/30

(52) U.S. Cl. CPC A61B 5/168; A61B 5/0048; A61B 5/1176; A61B 5/163; A61B 5/7264; A61B 5/742; G16H 10/20; G16H 50/30

(57) ABSTRACT

The present invention provides an intelligent attention rehabilitation system, which includes a gaze tracking module, an attention evaluation module, an intelligent computation and program push module, a data storage module, and an assessment and feedback module. In the invention, based on the theoretical CMA, data on attention level is acquired; and the advantages of simple configuration requirements and low cost of the gaze tracking technology, and intelligent computation of AI algorithms are used, to digitally evaluate attention of a subject. Besides, the DQN algorithm is used to realize intelligent push of related attention training guidance and programs, to solve the problem of family attention rehabilitation training under the lack of scientific guidance in the market currently, thereby providing a more scientific and accurate family attention rehabilitation evaluation and training system for the need.

### CROSS REFERENCE TO RELATED APPLICATION

This application is a continuation of U.S. application Ser. No. 17/155,002, entitled “Intelligent Attention Rehabilitation System” filed Jan. 21, 2021, and the entire contents of which are incorporated herein by reference.

### TECHNICAL FIELD

The disclosure relates to the field of Internet technologies, in particular to an intelligent attention rehabilitation system.

### BACKGROUND

An Attention Deficit Hyperactivity Disorder (ADHD), also known as hyperactivity, is a relatively common behavior disorder in childhood. Its clinical manifestations mainly include inattention, hyperactivity, impulsivity and other characteristics, accompanied by deviations in terms such as cognition, emotion, and behavior, which will severely affect the learning and life of children with ADHD, such as causing obvious learning disabilities, tensions with family members and children of the same age, lack of self-esteem, and coordination disorders. The prevalence of preschool children with ADHD in China is 4.31% to 5.83%. In recent years, data has shown that childhood ADHD can continue into adolescence or even adulthood, 30% to 70% of children with ADHD continue to have noticeable symptoms as adults, and their daily life and work will be affected. Therefore, it is particularly important to conduct scientific and accurate early screening and evaluation of attention disorders of children with ADHD, and to do intervention training in time. At present, the traditional attention rehabilitation evaluation is mainly based on scale evaluation and computer-assisted evaluation. There are limitations of the individual subjectivity of evaluators and the clinician's diagnosis and treatment experience in the scale evaluation. The evaluation needs to be carried out by a professional rehabilitation therapist or doctor, and the evaluation sites are mainly hospitals, rehabilitation medical institutions and special education institutions.

Attention, as a complex cognitive process, has been deeply studied by scholars from theoretical and clinical perspectives. At present, a Clinical Model of Attention (CMA) is classic and widely used, in which attention is divided into five dimensions: 1) focused attention, 2) sustained attention, 3) selective attention, 4) alternating attention, and 5) divided attention. The theoretical CMA is closely related to the individual's daily activities, has been proved to be useful in evaluating the attention of different pathological types, and is very suitable as a guiding theory for computer-assisted attention rehabilitation.

A gaze tracking method is an eye tracking technology that uses a common camera as an input device. No infrared transmitter is required, and users do not need to wear any device. Through designed algorithms, eye movements are tracked to acquire indexes such as gaze directions of eyes, duration of gaze and gaze points.

In aspects of an eye tracking technology and attention rehabilitation, it is found that, by searching for relevant research content, there is currently no attention rehabilitation system that combines the eye tracking technology. Therefore, it is of a great significance to use the gaze tracking technology combined with the CMA that is clinically practical to conduct careful research on all levels and dimensions of attention. A Deep Q-Network (DQN) algorithm can provide a technical support for establishment of a big database, intelligent analysis and comparison of data, and intelligent push of training programs and guidance.

Currently, there are some computer-assisted attention evaluation or training systems. In the patent “Method and System for Detecting and Training Attention Deficit Hyperactivity Disorder of Children Based on Virtual Reality”, with No. CN109350907, a detection method for ADHD is provided, using a virtual reality technology and an audio-visual integration continuous test technology to perform an ADHD test to a subject in a manner of games. In a test implementation process, Electroencephalogram (EEG) signals of the subject are required to be continuously acquired, and whether the subject is in a focused attention state or not is determined by reference with the EEG signals. But the virtual reality technology adopted by the system requires a large space and complicated connection lines, which is inconvenient to use. An EEG signal acquisition device may make a user uncomfortable during the test. Besides, after long-term virtual reality training, the child patient is prone to fatigue, and even has adverse reactions such as nausea and dizziness.

In the patent “Children Attention Evaluation System and Method Thereof”, with No. CN109646022, an attention evaluation model based on EEG is provided, in which a single electrode is fixed to the forehead of a user to acquire EEG of the forehead of a child and acquire original data of attention, and the original data of attention is processed and analyzed. The single electrode used in the system needs to be worn on the forehead of the subject, which increases discomfort of the patient and weakens experience feelings. Errors may be caused during the test due to turning of the patient's head.

In the patent “System and Method for Evaluating Children Attention”, with No. CN108665976, through a computer system provided with an input unit, a test unit, an evaluation unit, a recording unit, and the like, a subject is subjected to on-line and off-line tests of attention and evaluation of auditory perception and visual perception, and frequency analysis is performed by using a descriptive statistics method, followed by generation of evaluation reports. However, it needs to take 30-40 minutes for the system to complete all tests. For the subject with attention deficits, it is difficult to maintain a long-term attention state. The system relies on a computer and requires a readable storage medium containing specific computer programs, is not portable and is complicated in use, and cannot provide family training for the subject with attention deficits.

The off-line rehabilitation training mainly adopts biofeedback therapy and behavior modification therapy. Since the behavior modification therapy mainly adopts the form of one-to-one guidance and training, it needs the supervision and guidance of professional doctors or therapists, which may cause shortage and waste of children rehabilitation medical resources. Existing computer-assisted training systems are mainly for hospitals or special education institutions, can provide various assisted training for therapists, but are expensive and have a small audience. The training sites are mostly limited to hospitals or institutions, and family training and guidance for children with ADHD cannot be provided.

With the continuous development of science and technology, the computer-assisted training is a development trend of rehabilitation of children with ADHD. Methods based on the computer-assisted training such as a brain-computer interface and the virtual reality technology has been gradually applied in the field of ADHD treatment, making up for the shortcomings of the conventional treatment methods.

The brain-computer interface acquires a signal from the brain, analyzes and converts the signal into a command, and further converts the command into a signal from peripheral equipment, so as to provide a required output. Preliminary evaluation of children with ADHD may be achieved through specific evoked potentials. As a signal acquisition and processing technology, the brain-computer interface is low in hardware transmission efficiency, and has problems such as information delay and long response time in practical use, which affect user experiences. Feedback devices additionally arranged in a feedback system will greatly increase complexity of the system and affect a transmission rate of the system. In the meantime, the user experience will be weakened due to the complicated operation.

At present, the special attention rehabilitation system is relatively lacked in the market. Most of the existing systems use cognitive or speech rehabilitation systems as carriers, which are one of component modules in the systems. Take the attention training evaluation system, which is on sale, of Guangzhou Senku Medical Equipment Co., Ltd. as an example, the system is an internal module of the COGNI speech cognitive training system.

### SUMMARY

In view of the description of the Background, it is necessary for the disclosure to provide an intelligent attention rehabilitation system, which is combined with a gaze tracking device, is simple, does not need an external device to attach to the body of a child patient, can perform evaluation and training to the child patient in the most natural state in real time, and can achieve a more objective and accurate result.

In order to achieve the above purposes, the disclosure adopts the following solutions.

An intelligent attention rehabilitation system is provided. Modules of the system may implement quantitative analysis of data, include the following five modules: a gaze tracking module, an attention evaluation module, an intelligent computation and program push module, a data storage module, and an assessment and feedback module, and may achieve intelligent and personalized attention rehabilitation training for children. The gaze tracking module includes a server and a camera; the camera is used to acquire information of a facial image, and the server is used to position iris centers of human eyes according to the facial image. The attention evaluation module is used to evaluate focused attention, sustained attention, selective attention, alternating attention, divided attention, and Conners Parent Symptom Questionnaire (PSQ). The intelligent computation and program push module is used to compare and analyze evaluation scores of a subject to norm data in a database by receiving real-time data of the attention evaluation module and the gaze tracking module, and intelligently push an optimal training program through a Deep Q-Network (DQN) algorithm. The data storage module is used to receive data transmitted by the gaze tracking module and the attention evaluation module and data of intelligent training, and upload the data to the database. The assessment and feedback module is used for an operator of the system to check historical data of all users stored in the data storage module, and/or, to receive specific user information sent by the system.

The server of the gaze tracking module is particularly used for the following process.

At S1, information of the facial image is acquired through a common light-source-free single camera.

At S2, a position of a human face frame is detected by an Adaboost cascade algorithm.

At S3, facial feature points are calculated by a face alignment algorithm, to acquire an eye area image.

At S4, iris center detection is performed to the eye area image, a gray-scale differential on a circle of an iris image is calculated by a calculus operator, and a maximum value is taken from all differential results, so that iris centers of human eyes are accurately positioned.

At S5, coordinate positioning of the iris centers is performed.

In an equation of

$${max}_{( {x}_{0} , {y}_{0} , r )} \vert \frac{\partial }{\partial r} \frac{I ( x , y )}{2 π r} ds \vert ,$$

I (X, Y) is an image array, (x, Y) is the center of a circle, and r is a radius.

At S6, information of eye movement data, including gaze points, gaze duration, gaze frequency, and the time for gazing a stimulus point for the first time is acquired, an eye movement score is generated, and the eye movement score is transmitted to the attention evaluation module and the intelligent computation and program push module in real time.

Preferably, the eye movement score includes the time the subject stays at the stimulus point counted as a score a1, the frequency the subject gazes the stimulus point before completing a task counted as a score a2, and the time the subject gazes the stimulus point for the first time counted as a score a3; and the scores a1, a2 and a3 are added up to obtain the eye movement score A (a1+a2+a3=A).

Preferably, the camera is the light-source-free single camera.

Preferably, the attention evaluation module is used to display one or more preset visual stimulations through a display screen of the server, and provide a voice prompt required to be completed for the visual stimulations. The subject completes a corresponding task according to the prompt. The system may automatically generate corresponding attention scores according to task completion degree and time.

Particularly, the attention scores comprise:

A focused attention score B: select a specific number or character or symbol from randomly arranged stimuli of the same type within a limited time.

A sustained attention score C: delete as many specific targets as possible from randomly arranged stimuli of the same type within a limited time.

A selective attention score D: select a specific target from randomly arranged stimuli of various different types within a limited time.

An alternating attention score E: alternatively select a specific target, according to a voice prompt, from randomly arranged stimuli of two types within a limited time.

A divided attention score F: select a specific target from randomly arranged stimuli of the same type within a limited time, and tick a box if a specific syllable is heard during a task.

The Conners PSQ includes 48 items, and is completed by the father or the mother of a subject child. A questionnaire score G of 6 factors, including conduct problems, learning disorders, psychosomatic problems, impulsivity-hyperactivity, anxiety, and hyperactivity indexes, is obtained according to scores of the Conners PSQ.

Particularly, the intelligent computation and program push module intelligently pushes the optimal training program by using a Deep Q-Network (DQN) algorithm: the DQN algorithm continuously extracts data features from the database for learning, and through a large amount of data extraction and learning, the module learns experience and knowledge to realize selection and matching of training programs; and the intelligent computation and program push module automatically matches and adjusts difficulty and level of the next task according to a task completion status of the subject, provides a corresponding voice prompt, and conducts special training for project push programs with lower scores. For example, when the distance score a3 between a gaze point and a stimulus point of the subject is lower than the norm data, the system of the disclosure will lower the task completion standard; under the voice prompt, a task is considered completed if the gaze point of the subject is within a range of a circle that takes the stimulus as the center of a circle and has the radius of N cm, and the function is improved by gradually reducing the voice prompt and the radius of the circle.

The data storage module is particularly used to expand capacity of the database, which may include basic data on the attention level of normal children and children with different degrees of ADHD. Historical data of each evaluation and training of a user will be stored in a file of the subject. The same user may directly call the historical data when using the system.

Particularly, the operator of the system is a doctor or a therapist.

To sum up, the implementation of the system of the disclosure includes the following steps.
(1) After logging in to the system, the user enters basic information such as name, age, symptom duration and so on.
(2) The gaze tracking module tracks coordinate information of the iris centers of the subject in real time throughout the whole process, to acquire information of eye movement data, including gaze duration, gaze frequency, a distance between a gaze point and a stimulus, and the like, and to generate the eye movement score.
(3) The evaluation module gives the visual stimulations, and obtains corresponding attention scores based on the task completion status of the subject and the result of the Conners PSQ.
(4) The intelligent computation and program push module analyzes and compares the evaluation data, automatically generates the optimal training program, and intelligently adjusts the next training program according to the eye movement information and the task completion status during the training process.
(5) The data storage module stores data and uploads the data to the database to expand the capacity of the norm database. When the same user uses the system again, there is no need to evaluate again, and the historical data may be directly called to continue the next training program.
(6) The assessment and feedback module of the system selectively sends user data information to a background of a doctor port according to user scores, and reminds the doctor or the therapist to give advice and guidance within 2 working days.

Preferably, the specific user information includes user information (for example, user information with lower scores, A+B+C+D+E+F+G<XX) that the system will selectively send, according to the preset sending standards and user scores, to the background of the doctor port to remind the doctor or the therapist to give advice and guidance within 2 working days.

Compared with the conventional art, the disclosure has the advantages as followed.
(1) In the disclosure, use of multiple cameras or infrared light sources, as well as configuration calibration of the cameras, is not needed, the system has simple construction, and requirements for hardware or system configuration may be reduced.
(2) In the disclosure, real-time eye movement data of the subject may be acquired, and in conjunction with a theoretical Clinical Model of Attention (CMA) that is clinically practical, the attention level of the subject may be evaluated, which has high accuracy.
(3) In the disclosure, the system does not require any external device or invasive device, and is easy and safe to operate. The attention rehabilitation training may be completed individually or by accompanying of the parents of children with ADHD, so that the intelligent family attention rehabilitation training is achieved truly.
(4) In the disclosure, based on different attention levels of different users, data comparison is performed by means of database norms, the programs are intelligently pushed, and thus, intelligent and personalized training is provided.
(5) In the disclosure, according to the sending standards set by the system and the user scores, user data information will be selectively sent to the background of the doctor port. The doctor or the therapist will give advice and guidance within a limited time, which can be used for providing a more scientific training program for the attention rehabilitation training.

To sum up, in the disclosure, based on the theoretical CMA, data on attention level is acquired, and the advantages of simple configuration requirements and low cost of the gaze tracking technology, and intelligent computation of AI algorithms are used, to digitally evaluate attention of children with ADHD. Besides, the DQN algorithm is used to realize intelligent push of related attention training guidance and programs, to solve the problem of family attention rehabilitation training under the lack of scientific guidance in the market currently, thereby providing a more scientific and accurate family attention rehabilitation evaluation and training system for children with ADHD.

### BRIEF DESCRIPTION OF THE DRAWINGS

FIG. 1 is a flowchart of an implementation process of a system according to an embodiment of the disclosure;

FIG. 2 is a module schematic diagram of a system according to an embodiment of the disclosure; and

FIG. 3 is a module schematic diagram of an attention evaluation module according to an embodiment of the disclosure.

### DETAILED DESCRIPTION OF THE EMBODIMENTS

To make the implementation purposes, technical solutions and advantages of the disclosure clearer, the disclosure will be further described below in detail by referring to the accompanying drawings.

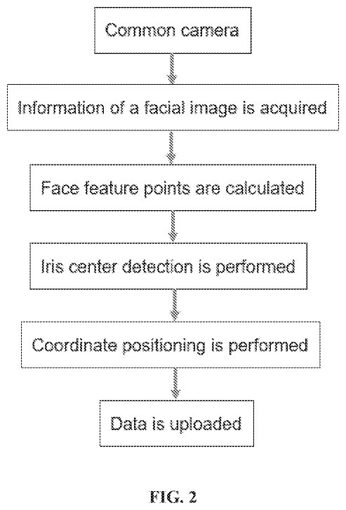

Embodiments of the disclosure provide an intelligent attention rehabilitation evaluation and training system, which uses gaze tracking and a Deep Q-Network (DQN) algorithm, and includes a gaze tracking module, an attention evaluation module, an intelligent computation and program push module, a data storage module, and an assessment and feedback module, as shown in FIG. 2.

The gaze tracking module includes a computer or a tablet computer, a common light-source-free single camera, without invasive devices.

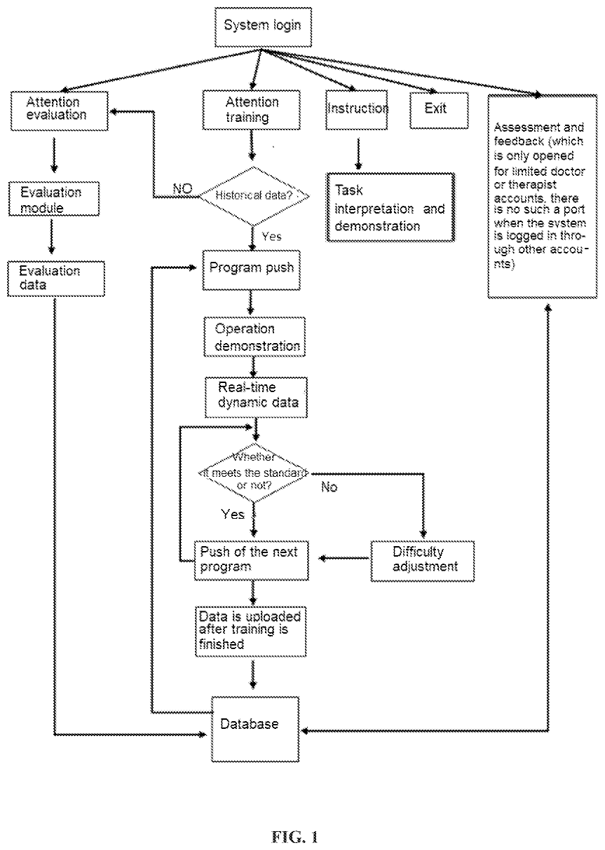

As shown in FIG. 1, the steps for using the gaze tracking module are as below.

At S1, information of a facial image is acquired through the common light-source-free single camera.

At S2, a position of a human face frame is detected by an Adaboost cascade algorithm. (Patent search results: Adaboost was used as the search term to acquire 4536 pieces of data, which directly used Adaboost, and the classifier algorithm was marked.)

At S3, facial feature points are calculated by a face alignment algorithm (a Supervised Descent Method, SDM), to acquire an eye area image. (Requirements: keep the head basically still or turn the head slightly at the angle less than 30 degrees)

At S4, iris center detection is performed to the eye area image, a gray-scale differential on a circle of an iris image is calculated by a calculus operator (a Daugman algorithm), and a maximum value is taken from all differential results, so that iris centers of human eyes are accurately positioned. (Requirements: good lighting conditions, and avoid using a template matching method for rough positioning, which has similar effects)

At S5, coordinate positioning of the iris centers is performed.

In an equation of

$${max}_{( {x}_{0} , {y}_{0} , r )} \vert \frac{\partial }{\partial r} \frac{I ( x , y )}{2 π r} ds \vert ,$$

I (X, Y) is an image array, (x, Y) is the center of circle, and r is a radius.

The source of functions and methods of the above-mentioned gaze tracking module is: “A Gaze Tracking Method Using Geometric Features of the Human Eyes” in Chinese Journal of Image and Graphics, published on June 2019.

The gaze tracking module acquires, by the above steps, information of eye movement data, including gaze points, gaze duration, gaze frequency, and the time for gazing a stimulus point for the first time, and generates an eye movement score. The eye movement score includes the time the subject stays at the stimulus point counted as a score a1, the frequency the subject gazes the stimulus point before completing a task counted as a score a2, and the time the subject gazes the stimulus point for the first time counted as a score a3; the scores a1, a2 and a3 are added up to obtain the eye movement score A (a1+a2+a3=A); and the eye movement score A is transmitted to the attention evaluation module and the intelligent computation and program push module in real time.

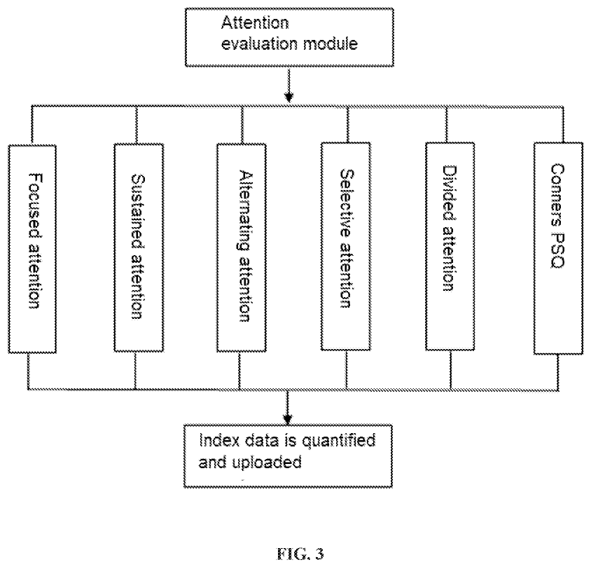

The attention evaluation module, as shown in FIG. 3, is based on the theory of a Clinical Model of Attention (CMA) that is classic and widely used clinically at present. Evaluation content includes six subtasks of focused attention, sustained attention, selective attention, alternating attention, divided attention, and Conners Parent Symptom Questionnaire (PSQ).

The evaluation module displays one or more preset visual stimulations through a display screen of the computer or the tablet computer, and provides a voice prompt required to be completed against the visual stimulations, and the subject is required to complete a corresponding task according to the prompt. According to the task completion degree and time, the system automatically generates corresponding attention scores. The attention scores comprise:
(1) A focused attention score B: select a specific number or character or symbols from randomly arranged stimuli of the same type within a limited time.
(2) A sustained attention score C: delete as many specific targets as possible from randomly arranged stimuli of the same type within a limited time.
(3) A selective attention score D: select a specific target from randomly arranged stimuli of various different types within a limited time.
(4) An alternating attention score E: alternatively select a specific target, according to a voice prompt, from randomly arranged stimuli of two types within a limited time.
(5) A divided attention score F: select a specific target from randomly arranged stimuli of the same type within a limited time, and tick a box if a specific syllable is heard during a task.
(6) The Conners PSQ includes 48 items, and is completed by the father or the mother of a subject child. A questionnaire score G of 6 factors, including conduct problems, learning disorders, psychosomatic problems, impulsivity-hyperactivity, anxiety, and hyperactivity indexes, is obtained according to scores of the Conners PSQ.

After the six subtasks are completed, all the evaluation data is transmitted to the intelligent computation and program push module.

The intelligent computation and program push module, by receiving real-time data of the attention evaluation module and the gaze tracking module, compares evaluation scores (A to G) of the subject to norm data of the database and analyzes the evaluation scores, and intelligently pushes an optimal training program through the DQN algorithm. The DQN algorithm continuously extracts data features from the database for learning, and through a large amount of data extraction and learning, the module learns experience and knowledge to realize selection and matching of training programs. The intelligent computation and program push module automatically matches and adjusts difficulty and level of the next task according to a task completion status of the subject, provides a corresponding voice prompt, and conducts special training for project push programs with lower scores. For example, when the distance score a3 between a gaze point and a stimulus point of the subject is lower than the norm data, the system of the disclosure will lower the task completion standard; under the voice prompt, a task is considered completed if the gaze point of the subject is within a range of a circle that takes the stimulus as the center of a circle and has the radius of N cm, and the function is improved by gradually reducing the voice prompt and the radius of the circle.

The data storage module is used to receive data of gaze tracking, attention evaluation and intelligent training, and upload the data to the database to expand capacity of the database, which may include basic data on the attention level of normal children and children with different degrees of ADHD. Historical data of each evaluation and training of a user will be stored in a file of the subject. The same user may directly call the historical data when using the system.

In use of the assessment and feedback module, an assessment and feedback module entry may be displayed when the system (i.e., a doctor port) is logged in through a specific account (the module is hidden and the entry is not displayed when other common users log in to the system), a doctor or a therapist may directly check historical data of all users in the data storage module through the doctor port. The system will selectively send user information (e.g., user information with low scores, A+B+C+D+E+F+G<XX), according to the preset sending standards and user scores, to the background of the doctor port, to remind the doctor or the therapist to give advice or guidance within 2 working days.

To sum up, the implementation of the system of the disclosure includes the following steps.
(1) After logging in to the system, the user enters basic information such as name, age, symptom duration and so on.
(2) The gaze tracking module tracks coordinate information of the iris centers of the subject in real time throughout the whole process, to acquire information of eye movement data, including gaze time, gaze frequency, a distance between a gaze point and a stimulus, and the like, and to generate the eye movement score.
(3) The evaluation module gives the visual stimulations, and obtains corresponding attention scores based on the task completion status of the subject and the result of the Conners PSQ.
(4) The intelligent computation and program push module analyzes and compares the evaluation data, automatically generates the optimal training program, and intelligently adjusts the next training program according to the eye movement information and the task completion status during the training process.
(5) The data storage module stores data and uploads the data to the database to expand the capacity of the norm database. When the same user uses the system again, there is no need to evaluate again, and the historical data can be directly called to continue the next training program.
(6) The assessment and feedback module of the system selectively sends user data information to the background of the doctor port according to user scores, and reminds the doctor or the therapist to give advice and guidance within 2 working days.

According to the disclosure, the intelligent attention rehabilitation evaluation and training system is provided. Evaluation and training indexes may be quantified based on the CMA in combination with the existing gaze tracking technology and the DQN algorithm. The attention index is assigned through the gaze tracking technology and scores of games that the user actively participates in, so that objective quantification of the attention evaluation index may be achieved. Children eye movement information may be dynamically monitored according to characteristics of children. Thus, accuracy and scientificity of attention evaluation and training for children with ADHD will be improved, and the scientific guidance for family training for children with ADHD may also be provided, thereby alleviating the shortage of children rehabilitation medical resources in China to a certain extent.

What is claimed is:

1. An intelligent attention rehabilitation system, comprising
a gaze tracking module,
an attention evaluation module,
an intelligent computation and program push module,
a data storage module, and
an assessment and feedback module, wherein
the gaze tracking module comprises a server and a camera, the camera is configured to acquire information of a facial image, and the server is configured to position iris centers of human eyes according to the facial image to generate an eye movement score A;
the attention evaluation module is configured to display one or more preset visual stimulations through a display screen of the sever, and provide a voice prompt required to be completed for the visual stimulations to the subject to complete a corresponding task according to the voice prompt, so that attention scores are generated according to task completion degree and time;
the intelligent computation and program push module is configured to compare and analyze an evaluation score of a subject to a norm data in a database by receiving real-time data of the attention evaluation module and the gaze tracking module, and push an optimal training program through a Deep Q-Network (DQN) algorithm; wherein the evaluation score is a sum of the eye movement score A and the attention scores;
the data storage module is configured to receive data transmitted by the gaze tracking module and the attention evaluation module and data of intelligent training, and upload the received data and the intelligent training data to the database; and
the assessment and feedback module is configured for an operator of the system to check historical data of all users stored in the data storage module, and/or, to receive a specific user information sent by the system.

2. The system according to claim 1, wherein the server of the gaze tracking module is configured to:
S1, detect a position of a human face frame by using an Adaboost cascade algorithm;
S2, calculate facial feature points by a face alignment algorithm, to acquire an eye area image;
S3, perform iris center detection to the eye area image, calculate a gray-scale differential on a circle of an iris image by a calculus operator, and take a maximum value from all differential results, so as to position the iris centers of human eyes;
S4, perform coordinate positioning of the iris centers, wherein
in an equation of
$${max}_{( {x}_{0} , {y}_{0} , r )} \vert \frac{\partial }{\partial r} \frac{I ( x , y )}{2 π r} ds \vert ,$$
I (x, y) is an image array, (x, y) is coordinate of the center of a circle, and r is a radius; and
S5, acquire information of eye movement data, comprising gaze points, a gaze duration the subject stays at a stimulus point, a gaze frequency the subject gazes the stimulus point before completing a task, and a time the subject gazes the stimulus point for a first time, generate the eye movement score A, and transmit the eye movement score A to the attention evaluation module and the intelligent computation and program push module in real time.

3. The system according to claim 2, wherein eye movement score A is a sum of score a1, score a2 and a score a3; wherein
the gaze duration the subject stays at the stimulus point is counted as the score a1, the gaze frequency the subject gazes the stimulus point before completing a task is counted as the score a2, the time the subject gazes the stimulus point for the first time is counted as the score a3.

4. The system according to claim 1, wherein the camera is a light-source-free single camera.

5. The system according to claim 2, wherein the camera is a light-source-free single camera.

6. The system according to claim 3, wherein the camera is a light-source-free single camera.

7. The system according to claim 1, wherein the attention scores comprise:
a focused attention score B evaluated by selecting a specific number or character or symbol from randomly arranged stimuli of the same type within a limited time;
a sustained attention score C evaluated by deleting as many specific targets as possible from randomly arranged stimuli of the same type within a limited time;
a selective attention score D evaluated by selecting a specific target from randomly arranged stimuli of various different types within a limited time;
an alternating attention score E evaluated by alternatively selecting a specific target, according to a voice prompt, from randomly arranged stimuli of two types within a limited time;
a divided attention score F evaluated by selecting a specific target from randomly arranged stimuli of the same type within a limited time, and tick a box if a specific syllable is heard during a task; and
a questionnaire score G obtained according to scores of a Conners Parent Symptom Questionnaire (PSQ); wherein
the Conners PSQ comprises 48 items, and is completed by the father or the mother of a subject child; the questionnaire score G comprises 6 factors, comprising conduct problems, learning disorders, psychosomatic problems, impulsivity-hyperactivity, anxiety, and hyperactivity indexes.

8. The system according to claim 7, wherein the intelligent computation and program push module intelligently pushes the optimal training program by using the DQN algorithm: the DQN algorithm continuously extracts data features from the database for learning, and through a large amount of data extraction and learning, the module learns experience and knowledge to realize selection and matching of training programs; the intelligent computation and program push module automatically matches and adjusts difficulty and level of the next task according to a task completion status of the subject in a task, provides a corresponding voice prompt, and conducts special training for project push programs with low evaluation score; wherein the low evaluation score is the evaluation score lower than the norm data in the database.

9. The system according to claim 1, wherein the data storage module is configured to expand capacity of the database, which comprises basic data on the attention level of normal children and children with different degrees of ADHD; historical data of each evaluation and training of a user will be stored in a file of the subject; and the same user may directly call the historical data when using the system.

10. The system according to claim 1, wherein the operator of the system is a doctor or a therapist.

11. The system according to claim 8, wherein the specific user information comprises user information selected according to preset sending standards and the evaluation scores; the assessment and feedback module is further configured to remind the doctor or the therapist to give advice and guidance within a set time.
